SEQUENCE LISTING (21 sequences)

SEQ ID NO: 1 - protein, 281 aa, Homo sapiens
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLARYR

SEQ ID NO: 2 - DNA, 345 bp, Mus musculus
caggtgaagctggatgagactggaggaggcttggtgcaacctgggaggcccatgaaactctcctgtgttgcctctggattcacttttagtgactactggatgaactgggtccgccagtctccagagaaaggactggagtgggtagcacaaattagagacaaaccttataattatgaaacattttattcagattctgtgaaaggcagattcaccatatcaagagatgattccaaaagtagtgtctacctgcaaatgaacaacttaggagctgaagacatgggtatctattactgtacagggtcgtttgcttactggggccaagggactctggtcactgtctctgca

SEQ ID NO: 3 - protein, 117 aa, Mus musculus
QVKLDETGGGLVQPGRPMKLSCVASGFTFSDYWMETNWVRQSPEKGLEWVAQIRDKPYNYETFYSDSVKGRFTISRDDSKSSVYLQMNNLGAEDMGIYYCTGSFAYWGQGTLVTVSA

SEQ ID NO: 4 - DNA, 45 bp, Artificial Sequence
  synthetic
ggtggcggtggttctggtggcggtggttctggtggcggtggttct

SEQ ID NO: 5 - protein, 15 aa, Artificial Sequence
  synthetic
GGGGSGGGGSGGGGS

SEQ ID NO: 6 - DNA, 324 bp, Mus musculus
gacatccagatgactcagtctccagcctccctatctgcatctgagggagaaactgtcaccatcacatgtcgagcaagtgggaatattcacaattatttagcatggtatcagcagaaacagggaaaatctcctcagctcctggtctataatgcaaaaaccttaccagatggtgtgccatcaagggtcagtggcagtggatcaggaacacagtattctctcaagatcaacaacctgcagcctgaagattttgggaattattactgccaacagtattggagtactccgtacacgttcggaggggggtccaggctggaaataaaacgg

SEQ ID NO: 7 - protein, 108 aa, Mus musculus
DIQMTQSPASLSASEGETVTITCRASGNIHNYLAWYQQKQGKSPQLLVYNAKTLPDGVPSRVSGSGSGTQYSLKINNLQPEDFGNYYCQQYWSTPYTFGGGSRLEIKR

SEQ ID NO: 8 - protein, 240 aa, Mus musculus
QVKLDETGGGLVQPGRPMKLSCVASGFTFSDYWMETNWVRQSPEKGLEWVAQIRDKPYNYETFYSDSVKGRFTISRDDSKSSVYLQMNNLGAEDMGIYYCTGSFAYWGQGTLVTVSAGGGGSGGGGSGGGGSDIQMTQSPASLSASEGETVTITCRASGNIHNYLAWYQQKQGKSPQLLVYNAKTLPDGVPSRVSGSGSGTQYSLKINNLQPEDFGNYYCQQYWSTPYTFGGGSRLEIKR

SEQ ID NO: 9 - DNA, 63 bp, Homo sapiens
atggccttaccagtgaccgccttgctcctgccgctggccttgctgctccacgccgccaggccg

SEQ ID NO: 10 - protein, 21 aa, Homo sapiens
MALPVTALLLPLALLLHAARP

SEQ ID NO: 11 - DNA, 147 bp, Homo sapiens
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagccc

SEQ ID NO: 12 - protein, 49 aa, Homo sapiens
KPTTTPAPRPPTPAPTIASQPLSLRPEASRPAAGGAVHTRGLDFASDKP

SEQ ID NO: 13 - DNA, 81 bp, Homo sapiens
ttttgggtgctggtggtggttggtggagtcctggcttgctatagcttgctagtaacagtggcctttattattttctgggtg

SEQ ID NO: 14 - protein, 27 aa, Homo sapiens
FWVLVVVGGVLACYSLLVTVAFIIFWV

SEQ ID NO: 15 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 16 - protein, 41 aa, Homo sapiens
RSKRSRLLHSDYMNMTPRRPGPTRKHYQPYAPPRDFAAYRS

SEQ ID NO: 17 - DNA, 342 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatag

SEQ ID NO: 18 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 493 aa, Homo sapiens
MALPVTALLLPLALLLHAARPASQVKLDETGGGLVQPGRPMKLSCVASGFTFSDYWMNWVRQSPEKGLEWVAQIRDKPYNYETFYSDSVKGRFTISRDDSKSSVYLQMNNLGAEDMGIYYCTGSFAYWGQGTLVTVSAGGGGSGGGGSGGGGSDIQMTQSPASLSASEGETVTITCRASGNIHNYLAWYQQKQGKSPQLLVYNAKTLPDGVPSRVSGSGSGTQYSLKINNLQPEDFGNYYCQQYWSTPYTFGGGSRLEIKRLEKPTTTPAPRPPTPAPTIASQPLSLRPEASRPAAGGAVHTRGLDFASDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 1449 bp, Homo sapiens
atggccttaccagtgaccgccttgctcctgccgctggccttgctgctccacgccgccaggccgcaggtgaagctggatgagactggaggaggcttggtgcaacctgggaggcccatgaaactctcctgtgttgcctctggattcacttttagtgactactggatgaactgggtccgccagtctccagagaaaggactggagtgggtagcacaaattagagacaaaccttataattatgaaacattttattcagattctgtgaaaggcagattcaccatatcaagagatgattccaaaagtagtgtctacctgcaaatgaacaacttaggagctgaagacatgggtatctattactgtacagggtcgtttgcttactggggccaagggactctggtcactgtctctgcaggtggcggtggttctggtggcggtggttctggtggcggtggttctgacatccagatgactcagtctccagcctccctatctgcatctgagggagaaactgtcaccatcacatgtcgagcaagtgggaatattcacaattatttagcatggtatcagcagaaacagggaaaatctcctcagctcctggtctataatgcaaaaaccttaccagatggtgtgccatcaagggtcagtggcagtggatcaggaacacagtattctctcaagatcaacaacctgcagcctgaagattttgggaattattactgccaacagtattggagtactccgtacacgttcggaggggggtccaggctggaaataaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 482 aa, Homo sapiens
MALPVTALLLPLALLLHAARPQVKLDETGGGLVQPGRPMKLSCVASGFTFSDYWMNWVRQSPEKGLEWVAQIRDKPYNYETFYSDSVKGRFTISRDDSKSSVYLQMNNLGAEDMGIYYCTGSFAYWGQGTLVTVSAGGGGSGGGGSGGGGSDIQMTQSPASLSASEGETVTITCRASGNIHNYLAWYQQKQGKSPQLLVYNAKTLPDGVPSRVSGSGSGTQYSLKINNLQPEDFGNYYCQQYWSTPYTFGGGSRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2021/0340271 A1
(43) Pub. Date: Nov. 4, 2021

(54) CD37-ANTIBODY AND CD37-CAR-T CELLS

(71) Applicants: ProMab Biotechnologies, Inc., Richmond, CA (US); Forevertek Biotechnology Co., Ltd, Changsha (CN)

(72) Inventors: Lijun Wu, Albany, CA (US); Vita Golubovskaya, Richmond, CA (US)

(21) Appl. No.: 17/371,002

(22) Filed: Jul. 8, 2021

Related U.S. Application Data
(63) Continuation of application No. PCT/US2020/012385, filed on Jan. 6, 2020.
(60) Provisional application No. 62/791,173, filed on Jan. 11, 2019.

(51) Int. Cl. C07K 16/28; C07K 14/705; C07K 14/725

(52) U.S. Cl. CPC C07K 16/2896; C07K 14/70521; C07K 2319/03; C07K 14/7051; C07K 2317/622; C07K 14/70578

(57) ABSTRACT

The present invention is directed to a monoclonal anti-human CD37 antibody or a single-chain variable fragment (scFv), comprising V_{H} having the amino acid of SEQ ID NO: 3 and V_{L} having the amino acid of SEQ ID NO: 7. The present invention is also directed to a chimeric antigen receptor fusion protein comprising from N-terminus to C-terminus: (i) a single-chain variable fragment (scFv) of the present invention, (ii) a transmembrane domain, (iii) at least one co-stimulatory domains, and (iv) an activating domain.

This application is a continuation of PCT/US2020/012385, filed Jan. 6, 2020; which claims the priority of U.S. Provisional Application No. 62/791,173, filed Jan. 11, 2019. The contents of the above-identified applications are incorporated herein by reference in their entireties.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Dec. 19, 2019, and a size of 8 kilobytes. The Sequence Listing filed via EFS-Web is part of the specification and is hereby incorporated in its entirety by reference herein.

### FIELD OF THE INVENTION

The present invention relates to CD37-specific antibody and CD37-CAR-T Cells, which are useful in the field of adoptive immunity gene therapy for tumors.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Genetically modifying T cells with CAR (Chimeric antigen receptor) constructs is the most common approach to design tumor-specific T cells. CAR-T cells targeting tumor-associated antigens (TAA) can be infused into patients (called adoptive cell transfer or ACT) representing an efficient immunotherapy approach [1, 2]. The advantage of CAR-T technology compared with chemotherapy or antibody is that reprogrammed engineered T cells can proliferate and persist in the patient (“a living drug”) [1, 3].

CARs typically consist of a monoclonal antibody-derived single-chain variable fragment (scFv) at the N-terminal part, hinge, transmembrane domain and a number of intracellular co-activation domains: (i) CD28, (ii) CD137 (4-1BB), CD27, or other co-stimulatory domains, in tandem with an activation CD3-zeta domain. (FIG. 1) [1,2]. The evolution of CARs went from first generation (with no co-stimulation domains) to second generation (with one co-stimulation domain) to third generation CAR (with several co-stimulation domains). Generating CARs with two costimulatory domains (the so-called 3rd generation CAR) have led to increased cytolytic CAR-T cell activity, improved persistence of CAR-T cells leading to its augmented antitumor activity.

FIG. 1 illustrates the structures of CAR. The left panel shows the structure of the first generation of CAR (no costimulatory domains). The middle panel shows the structure of the second generation of CAR (one co-stimulation domain CD28 or 4-BB). The right panel shows the third generation of CAR (two or several co-stimulation domains) [6].

Natural killer cells, or NK cells, are a type of cytotoxic lymphocyte critical to the innate immune system. The role NK cells play is analogous to that of cytotoxic T cells in the vertebrate adaptive immune response. NK cells provide rapid responses to virus-infected cells, acting at around 3 days after infection, and respond to tumor formation.

CD37 antigen (or Tetraspanin-26, Tspan-26) is a cell surface glycoprotein that is encoded by CD37 gene. Tetraspanins are known for four transmembrane regions, and 2 extracellular loops. The CD37 protein is 281 amino-acid protein, with 39-59 amino-acids and 112-241 amino-acids representing extracellular regions of protein.

CD37 expression is restricted to lymphoid tissues, and in particular to mature B cells, with low levels of expression on plasma cells and dendritic cells. CD37 is expressed in mature B-cell neoplasms including mantle cell lymphoma, follicular lymphoma, diffuse large B-cell lymphoma, Burkitt's lymphoma, and chronic lymphocytic leukemia, whereas it is low or absent in acute lymphoblastic leukemia and multiple myeloma. [7]

### BRIEF DESCRIPTION OF THE DRAWINGS

FIG. 1 shows the structures of CAR.

FIG. 2 shows the amino acid sequence of CD37 protein (SEQ ID NO: 1). Two extracellular domains are underlined and in bold.

FIG. 3 shows the structure of CD37 CAR construct. The second-generation CAR is used with either CD28 or 41BB as a co-stimulatory domain.

FIG. 4A demonstrates specific immunofluorescent staining of the CD37 antibody (top panel) with HEK293-CD37, but not with HEK293, HEK293-CD18. The bottom panel shows DAPI (4′,6-diamidino-2-phenylindole) blue-fluorescent staining of the nucleus DNA, which demonstrates the presence of the cells. FIG. 4B shows that CD37 antibody detected CD37 antigen in CHO-CD37 cells (right panel) but not in CHO cells (left panel). The middle panel used an isotype antibody as a control. FIG. 4C shows CD37 antibody detected CD37 antigen in body-cavity-based lymphoma (BCBL-1) cell line (left panel) and mouse lymphocytic leukemia cell line L1210 cell line (right panel).

FIG. 5 shows FACS staining on CD37 positive Raji lymphoma cells, but no staining or a very weak staining in CD37 negative cells such as Lovo colon cancer cells, MCF-7, MDA-231 breast cancer cells, K562 chronic myelogenous leukemia cells, and RPMI8226 multiple myeloma cells.

FIG. 6 shows that FACS with mouse FAB antibody detects CD37+CAR+ positive cells after transduction with CD37-28-CD3 CAR virus. Left panel: Control T cells. Right panel: CD37-CAR positive cells.

FIGS. 7A-7C are real-time RTCA assay results demonstrating specific killing activity of CD37-CAR-T cells against CHO-CD37 target cells but not CHO cells.

FIG. 8 shows high IFN-gamma secretion by CD37-CAR-T cells against CHO-CD37 cells, but not by CD37-CAR-T cells against CHO cells. p<0.05 for both *CD37-CD28-CAR-T, **CD37-41BB-CAR-T cells, versus mock-CAR-T cells, Student's t-test.

FIG. 9 shows significantly higher secretion of IFN-gamma with CD37-CD28-CAR-T cells against Raji target cells than with CD37-CD28-CAR-T cells against K562 cells. *p<0.05, CD37-28-CD3 in Raji cells versus T or K562 cells by Student's t-test.

FIG. 10 shows images of Raji xenografts in NSG mice after treatment with CD37-CAR-T cells, PBS, and T cells. Left panel shows mice had decreased signals after treating with CD37-28-CD3 CAR-T cells. Right panel shows total flux (Y-axis: photons/sec; X-axis: days after Raji cells injection into NSG mice). *p<0.05 CD37-CD28-CD3 cells vs. T cells at day 14.

FIG. 11 is a Kaplan-Meier curve, which shows increased survival of CD37-CAR-T cell-treated mice in Raji xenograft in vivo model. *p<0.05, CD37-CAR-T cells versus PBS control, Student's t-test.

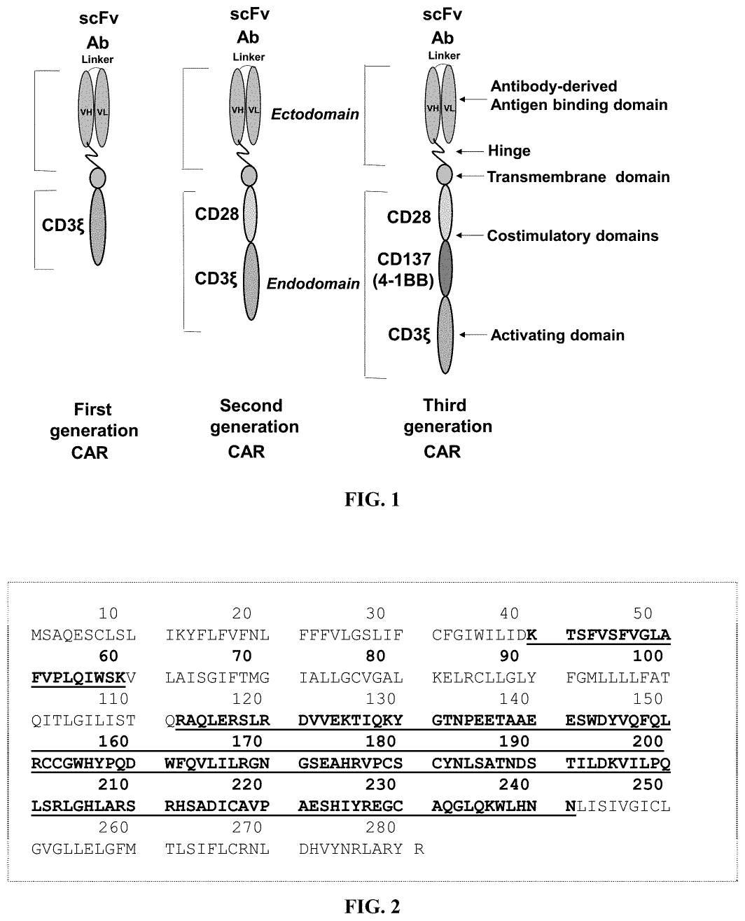

### DETAILED DESCRIPTION OF THE INVENTION

### Definitions

As used herein, a “chimeric antigen receptor (CAR)” is a receptor protein that has been engineered to give T cells the new ability to target a specific protein. The receptor is chimeric because they combine both antigen-binding and T-cell activating functions into a single receptor. CAR is a fused protein comprising an extracellular domain capable of binding to an antigen, a transmembrane domain, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell. As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for engineering an scFv are known to a person skilled in the art.

As used herein, a “tumor antigen” means a biological molecule having antigenecity, expression of which causes cancer.

The inventors have generated mouse monoclonal antibody specifically targeting human CD37. The present invention is directed to a monoclonal anti-human CD37 antibody comprising V_{H} having the amino acid of SEQ ID NO: 3 and V_{L} having the amino acid of SEQ ID NO: 7. The monoclonal anti-human CD37 antibody was generated against the extracellular region of the purified recombinant fragment of human CD37 (110-241 amino-acids). In one embodiment, the monoclonal anti-human CD37 antibody is a single-chain variable fragment (scFv). ScFv can be V_{H}-linker-V_{L} or V_{L}-linker-V_{H}.

The present invention is also directed to a chimeric antigen receptor fusion protein comprising from N-terminus to C-terminus: (i) a single-chain variable fragment (scFv) against CD37, in which V_{H} has the amino acid sequence of SEQ ID NO: 3, and V_{L} has the amino acid of SEQ ID NO: 7, (ii) a transmembrane domain, (iii) at least one co-stimulatory domains, and (iv) an activating domain. The CD37-CAR-T cells of the present invention have high cytotoxic activity against several cancer cell lines. The inventors have produced CD37-CAR-T cells to target cancer cells overexpressing CD37 tumor antigen.

In one embodiment, the CAR structure is shown in FIG. 2.

In one embodiment, the co-stimulatory domain is selected from the group consisting of CD28, 4-1BB, GITR, ICOS-1, CD27, OX-40 and DAP10. A preferred the co-stimulatory domain is CD28 or 4-1BB.

A preferred activating domain is CD3 zeta (CD3 Z or CD3ζ).

The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3 zeta chain, CD28, CD3ε, CD45, CD4, CDS, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. Optionally, a short oligopeptide linker or a polypeptide linker, for example, a linker having a length of 2 to 10 amino acids can be arranged between the transmembrane domain and the intracellular domain. In one embodiment, a linker sequence having a glycine-serine continuous sequence can be used.

The present invention provides a nucleic acid encoding the CD37-CAR. The nucleic acid encoding the CAR can be prepared from an amino acid sequence of the specified CAR by a conventional method. A base sequence encoding an amino acid sequence can be obtained from the aforementioned NCBI RefSeq IDs or accession numbers of GenBank for an amino acid sequence of each domain, and the nucleic acid of the present invention can be prepared using a standard molecular biological and/or chemical procedure. For example, based on the base sequence, a nucleic acid can be synthesized, and the nucleic acid of the present invention can be prepared by combining DNA fragments which are obtained from a cDNA library using a polymerase chain reaction (PCR).

A nucleic acid encoding the CAR of the present invention can be inserted into a vector, and the vector can be introduced into a cell. For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a sendai virus vector, an Epstein-Barr virus (EBV) vector, and a HSV vector can be used. A virus vector lacking the replicating ability so as not to self-replicate in an infected cell is preferably used.

For example, when a retrovirus vector is used, a suitable packaging cell based on a LTR sequence and a packaging signal sequence possessed by the vector can be selected for preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies.

A CAR-T cell binds to a specific antigen via the CAR, thereby a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, or the like as an index. For example, release of a cytotoxic cytokine (IFN-gamma, a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and a macrophage.

The cell expressing the CAR can be used as a therapeutic agent for a disease. The therapeutic agent comprises the cell expressing the CAR as an active ingredient, and it may further comprise a suitable excipient.

The inventors have generated CD37-CAR-T cells against hematological cancer cells overexpressing CD37. The inventors have provided data demonstrating efficient expression of CD37 in hematological cancers cancer such as lymphoma and certain leukemia. CD37-CAR-T cells express higher cytotoxic activity against CD37-positive cancer cells than against non-transduced T cells and Mock-CAR-T cells.

The advantage of the CD37 monoclonal antibody or CD37-ScFv of the present invention over other known CD37 antibodies is that the present antibody has high binding activity to lymphoma antigen, and it is highly specific against CD37-positive cancer cells of lymphoma. This provides a wider range of the antibody application for targeting hematological cancers. The CD37 antibody is highly potent as a therapeutic agent in many clinical applications.

The present monoclonal mouse anti-human CD37 antibody detects CD37 in CD37-positive cancer cells.

The present CD37 antibody can be used for immunotherapy applications: toxin/drug-conjugated Ab, monoclonal therapeutic antibody, humanization of CD37 antibody, and CAR-T cell immunotherapy.

CD37-CAR-T cells using the present CD37 antibody can target CD37 antigen in CD37-positive cell line such as lymphoma, and certain leukemia.

CD37-CAR-T can be used in combination with different therapies: checkpoint inhibitors; targeted therapies, small molecule inhibitors, and antibodies.

CD37 antibody can be modified with site-directed mutagenesis for affinity tuning; it can be humanized and can be used for a complete human antibody generation.

CD37-CAR-T cells can be used clinically for CD37-positive cells.

Modifications of co-stimulating domains of CD28 or 4-1BB may increase the efficacy of CAR. Tag-conjugated CD37 scFv can be used for CAR generation.

Third generation CAR-T or other co-activation signaling domains can be used with the present CD37-scFv to prepare CD37-CAR.

The present mouse CD37 antibody can be humanized for generation of CD37-CAR.

Combination of CD37-CAR with other CAR targeting other tumor antigens or tumor microenvironment (VEGFR-1-3), PDL-1, CD80, or bi-scFv-CAR can be used to enhance activity of monotherapy CD37-CAR. Bi-specific antibodies of CD37 and CD3 or other antigens can be generated for therapy.

The present CD37-CAR can be used to generate other types of cells such as CAR-natural killer (NK) cells, CD37-CAR-macrophages, and other CD37-CAR hematopoietic cells, which can target CD37-positive cancers. The present invention provides T cells, or NK cells, or macrophages, or hematopoietic cells, modified to express the CD37-CAR.

The following examples further illustrate the present invention. These examples are intended merely to be illustrative of the present invention and are not to be construed as being limiting.

### EXAMPLES

### Example 1. Anti-CD37 Antibody: V_{H} and V_{L} and scFv Sequences

We generated mouse monoclonal anti-human CD37 antibody using a standard hybridoma technology as described by Boeye [4]. CD37 scFv was obtained by sequencing one of the hybridoma clones (2B8D12)-positive for CD37. The structure of CD37 scFv is: V_{H}-linker-V_{L}.

The nucleotide sequence and the amino acid sequence of mouse CD37 V_{H} are shown below.

(SEQ ID NO: 2)
CAGGTGAAGCTGGATGAGACTGGAGGAGGCTTGGTGCAACCTGGGAGGCC
CATGAAACTCTCCTGTGTTGCCTCTGGATTCACTTTTAGTGACTACTGGA
TGAACTGGGTCCGCCAGTCTCCAGAGAAAGGACTGGAGTGGGTAGCACAA
ATTAGAGACAAACCTTATAATTATGAAACATTTTATTCAGATTCTGTGAA
AGGCAGATTCACCATATCAAGAGATGATTCCAAAAGTAGTGTCTACCTGC
AAATGAACAACTTAGGAGCTGAAGACATGGGTATCTATTACTGTACAGGG
TCGTTTGCTTACTGGGGCCAAGGGACTCTGGTCACTGTCTCTGCA
(SEQ ID NO: 3)
Q V K L D E T G G G L V Q P G R P M K L S C V A S
G F T F S D Y W Met N W V R Q S P E K G L E W V A
Q I R D K P Y N Y E T F Y S D S V K G R F T I S R
D D S K S S V Y L Q M N N L G A E D M G I Y Y C T
G S F A Y W G Q G T L V T V S A

The nucleotide sequence and the amino acid sequence of a linker are shown below.

(SEQ ID NO: 4)
GGTGGCGGTGGTTCT GGTGGCGGTGGTTCT GGTGGCGGTGGTTCT
(SEQ ID NO: 5)
G G G G S G G G G S G G G G S

The nucleotide sequence and the amino acid sequence of mouse CD37 V_{L} are shown below.

(SEQ ID NO: 6)
GACATCCAGATGACTCAGTCTCCAGCCTCCCTATCTGCATCTGAGGGAGA
AACTGTCACCATCACATGTCGAGCAAGTGGGAATATTCACAATTATTTAG
CATGGTATCAGCAGAAACAGGGAAAATCTCCTCAGCTCCTGGTCTATAAT
GCAAAAACCTTACCAGATGGTGTGCCATCAAGGGTCAGTGGCAGTGGATC
AGGAACACAGTATTCTCTCAAGATCAACAACCTGCAGCCTGAAGATTTTG
GGAATTATTACTGCCAACAGTATTGGAGTACTCCGTACACGTTCGGAGGG
GGGTCCAGGCTGGAAATAAAACGG
(SEQ ID NO: 7)
D I Q M T Q S P A S L S A S E G E T V T I T C R A
S G N I H N Y L A W Y Q Q K Q G K S P Q L L V Y N
A K T L P D G V P S R V S G S G S G T Q Y S L K I
N N L Q P E D F G N Y Y C Q Q Y W S T P Y T F G G
G S R L E I K R

The amino acid sequence of CD37 scFv (V_{H}-linker-V_{L}) is shown below. The bold highlights the amino acid sequence of V_{H} (SEQ ID NO: 3); the underlined highlights the amino sequence of V_{L} (SEQ ID NO: 7); in between (italicized) is the amino acid sequence of 3xG4S linker sequence (SEQ ID NO: 5).

(SEQ ID NO: 8)
Q V K L D E T G G G L V Q P G R P M K L S C V A S
G F T F S D Y W Met N W V R Q S P E K G L E W V A
Q I R D K P Y N Y E T F Y S D S V K G R F T I S R
D D S K S S V Y L Q M N N L G A E D M G I Y Y C T
G S F A Y W G Q G T L V T V S A G G G G S G G G G
S G G G G S D I Q M T Q S P A S L S A S E G E T V
T I T C R A S G N I H N Y L A W Y Q Q K Q G K S P
Q L L V Y N A K T L P D G V P S R V S G S G S G T
Q Y S L K I N N L Q P E D F G N Y Y C Q Q Y W S T
P Y T F G G G S R L E I K R

### Example 2. CD37 Antibody Detected CD37 Protein in CD37-Positive cells by Immunostaining

The antibody CD37, clone 2B8D12, detected extracellular CD37 protein by ELISA (0D405 reading with CD37 protein was 1.53, while with negative unrelated control protein was 0.098). The antibody is IgG1 type.

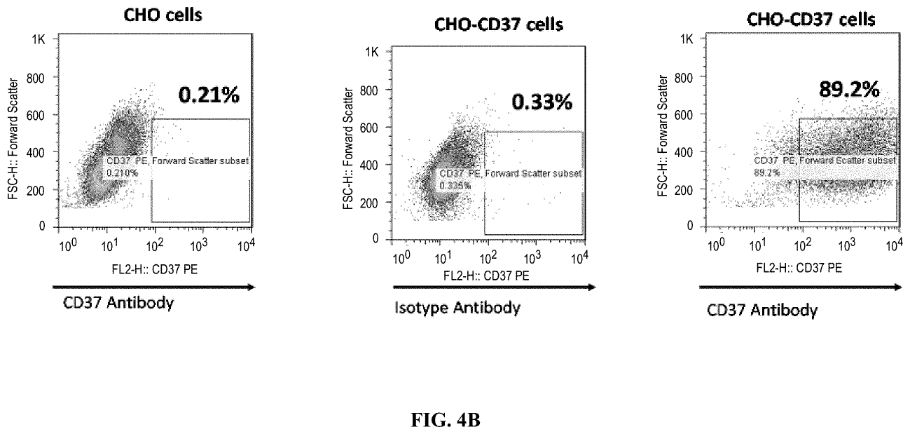

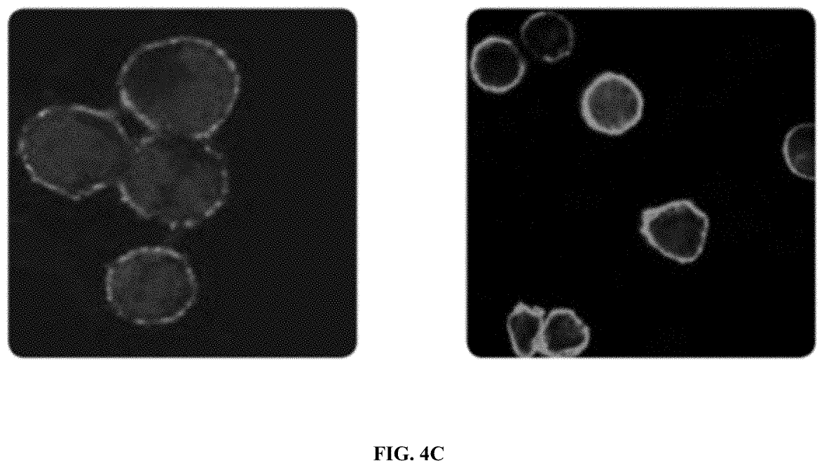

FIG. 4A demonstrates specific immunofluorescent staining of the CD37 antibody (top panel) with HEK 293 cell line transformed with CD37-containing plasmid (HEK293-CD37), but not with non-transfected HEK293 (HEK293), or HEK 293 cell line transformed with a negative control protein CD18 (HEK293-CD18). The bottom panel shows DAPI (4′,6-diamidino-2-phenylindole) blue-fluorescent staining of the nucleus DNA to show the presence of the cells.

FIG. 4B shows CD37 antibody detected CD37 antigen in CHO-CD37 cells (right panel) having a stable expression of CD37, but not in CHO cells (left panel). The middle panel used an isotype antibody as a control.

FIG. 4C shows CD37 antibody detected CD37 antigen in body-cavity-based lymphoma (BCBL-1) cell line (left panel) and mouse lymphocytic leukemia cell line L1210 cell line (right panel). Immunostaining in both cell lines showed membrane staining.

### Example 3. Low Expression of CD37 in Most Normal Tissues by IHC Staining

The immunohistochemical staining with CD37 antibody show negative staining in most normal tissues but demonstrated very high expression in tonsils where hematological cells are. Dilution of CD37 antibody (2B8D12) was 1:700. There was some positive staining in liver stomach, duodenum and breast cancer. Most tissues were negative: esophagus, esophageal carcinoma, thyroid, colon cancer, rectum, testicle, and other tissues.

### Example 4. CD37 Antibody Detected CD37 in CD37-Positive Lymphoma Cells

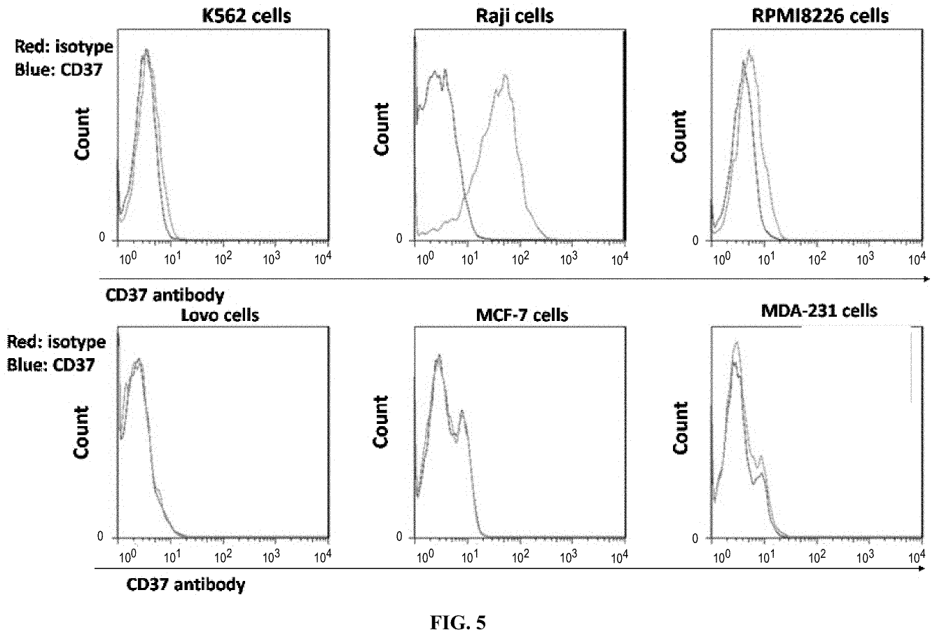

We stained different cell lines with the CD37 antibody. FIG. 5 shows FACS staining on CD37 positive Raji lymphoma cells, but negative in CD37 negative cells such as Lovo colon cancer cells, MCF-7, MDA-231 breast cancer cells, K562 chronic myelogenous leukemia cells, and very weak staining in CD37 negative RPMI8226 multiple myeloma cells. K562 chronic myelogenous leukemia cells do not express CD37.

### Example 5. CD37-CAR Sequences

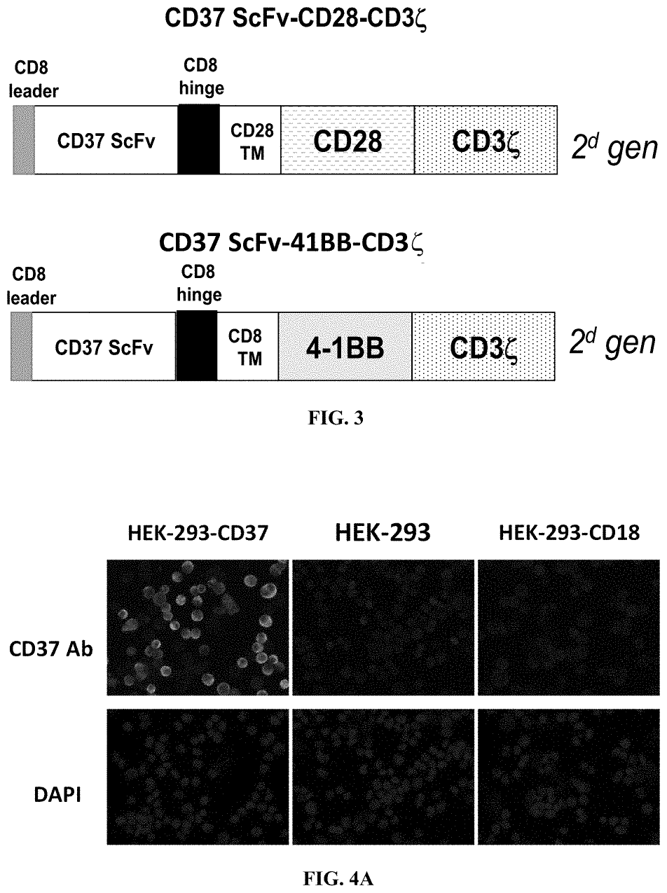

The scheme of CD37-CAR construct is shown on FIG. 3. Lentiviral vector Lentiviral vector with EF1 promoter was used for cloning of the scFv CAR sequence. We also used MNDU-3 promoter lentiviral vector to increase percent of CAR-positive cells.

The following nucleotide sequence shows CD37 ScFv -CD8 hinge-TM28-CD28-CD3 zeta of the present invention. The structure includes human CD8 signaling peptide, mouse CD37 scFv (V_{H}-Linker 3x(G4S)-V_{L}), human CD8 hinge, human CD28 transmembrane, co-stimulating domain human CD28, activation domain human CD3 zeta (FIG. 3).

The nucleic acid sequence and amino acid sequence of each segment of CD8 leader-CD37 scFv (V_{H}-Linker-V_{L})-CD8 hinge-CD28 TM-CD28-CD3-zeta (CD37-CD28 CAR) are shown below.

<CD8 leader>
(SEQ ID NO: 9)
ATGGCCTTACCAGTGACCGCCTTGCTCCTGCCGCTGGCCTTGCTGCTCCA
CGCCGCCAGGCCG
(SEQ ID NO: 10)
M A L P V T A L L L P L A L L L H A A R P
<Nhe I site>
GCTAGC
<CD37 ScFv> V_{H}-linker-V_{L}

See Example 1 for nucleic acid sequences and amino acid sequences.

<XhoI restriction site>
CTCGAG
<CD8 hinge>
(SEQ ID NO: 11)
AAGCCCACCACGACGCCAGCGCCGCGACCACCAACACCGGCGCCCACCA
TCGCGTCGCAGCCCCTGTCCCTGCGCCCAGAGGCGAGCCGGCCAGCGGC
GGGGGGCGCAGTGCACACGAGGGGGCTGGACTTCGCCAGTGATaagccc
(SEQ ID NO: 12)
K P T T T P A P R P P T P A P T I A S Q P L S L R
P E A S R P A A G G A V H T R G L D F A S D K P
<CD28 TM>
(SEQ ID NO: 13)
TTTTGGGTGCTGGTGGTGGTTGGTGGAGTCCTGGCTTGCTATAGCTTGC
TAGTAACAGTGGCCTTTATTATTTTCTGGGTG
(SEQ ID NO: 14)
F W V L V V V G G V L A C Y S L L V T V A F I I F
W V
<CD28/Co-stimulation domain>
(SEQ ID NO: 15)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC
(SEQ ID NO: 16)
R S K R S R L L H S D Y M N M T P R R P G P T R K
H Y Q P Y A P P R D F A A Y R S
<CD3 zeta>
(SEQ ID NO: 17)
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAATAG
(SEQ ID NO: 18)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
<EcoRI restriction site>
gaattc

The amino-acid sequence of CD37-CAR protein is shown below.

(SEQ ID NO: 19)
M A L P V T A L L L P L A L L L H A A R P A S Q V
K L D E T G G G L V Q P G R P M K L S C V A S G F
T F S D Y W M N W V R Q S P E K G L E W V A Q I R
D K P Y N Y E T F Y S D S V K G R F T I S R D D S
K S S V Y L Q M N N L G A E D M G I Y Y C T G S F
A Y W G Q G T L V T V S A G G G G S G G G G S G G
G G S D I Q M T Q S P A S L S A S E G E T V T I T
C R A S G N I H N Y L A W Y Q Q K Q G K S P Q L L
V Y N A K T L P D G V P S R V S G S G S G T Q Y S
L K I N N L Q P E D F G N Y Y C Q Q Y W S T P Y T
F G G G S R L E I K R L E K P T T T P A P R P P T
P A P T I A S Q P L S L R P E A S R P A A G G A V
H T R G L D F A S D K P F W V L V V V G G V L A C
Y S L L V T V A F I I F W V R S K R S R L L H S D
Y M N M T P R R P G P T R K H Y Q P Y A P P R D F
A A Y R S R V K F S R S A D A P A Y Q Q G Q N Q L
Y N E L N L G R R E E Y D V L D K R R G R D P E M
G G K P Q R R K N P Q E G L Y N E L Q K D K M A E
A Y S E I G M K G E R R R G K G H D G L Y Q G L S
T A T K D T Y D A L H M Q A L P P R

Similar CD37-CAR construct was prepared with CD8 transmembrane domain and 4-1BB co-stimulatory domains instead of CD28TM, and CD28 co-stimulating domains, respectively.

CD8 leader-CD37 scFv (V_{H}-Linker-V_{L})-CD8 hinge-CD8 TM-4-1BB-CD3 zeta (CD37-4-1BB CAR):

Nucleotide sequence
(SEQ ID NO: 20)
ATGGCCTTACCAGTGACCGCCTTGCTCCTGCCGCTGGCCTTGCTGCTCCACGCCGCCAGGCCG
CAGGTGAAGCTGGATGAGACTGGAGGAGGCTTGGTGCAACCTGGGAGGCCCATGAA
ACTCTCCTGTGTTGCCTCTGGATTCACTTTTAGTGACTACTGGATGAACTGGGTCCGC
CAGTCTCCAGAGAAAGGACTGGAGTGGGTAGCACAAATTAGAGACAAACCTTATAA
TTATGAAACATTTTATTCAGATTCTGTGAAAGGCAGATTCACCATATCAAGAGATGA
TTCCAAAAGTAGTGTCTACCTGCAAATGAACAACTTAGGAGCTGAAGACATGGGTA
TCTATTACTGTACAGGGTCGTTTGCTTACTGGGGCCAAGGGACTCTGGTCACTGTCTCTGCA
GGTGGCGGTGGTTCT GGTGGCGGTGGTTCT GGTGGCGGTGGTTCT
GACATCCAGATGACTCAGTCTCCAGCCTCCCTATCTGCATCTGAGGGAGAAACTGTC
ACCATCACATGTCGAGCAAGTGGGAATATTCACAATTATTTAGCATGGTATCAGCAG
AAACAGGGAAAATCTCCTCAGCTCCTGGTCTATAATGCAAAAACCTTACCAGATGGT
GTGCCATCAAGGGTCAGTGGCAGTGGATCAGGAACACAGTATTCTCTCAAGATCAA
CAACCTGCAGCCTGAAGATTTTGGGAATTATTACTGCCAACAGTATTGGAGTACTCC
GTACACGTTCGGAGGGGGGTCCAGGCTGGAAATAAAACGG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ATCTACATCTGGGCGCCCCTGGCCGGGACTTGTGG
GGTCCTTCTCCTGTCACTGGTTATCACCCTTTACTGCAAACGGGGCAGAAAGAAACTCC
TGTATATATTCAAACAACCATTTATGAGACCAGTACAAACTACTCAAGAGGAAGATGGCTGT
AGCTGCCGATTTCCAGAAGAAGAAGAAGGAGGATGTGAACTGAGAGTGAAGTTCAGCA
GGAGCGCAGACGCCCCCGCGTACAAGCAGGGCCAGAACCAGCTCTATAACGAG
CTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTG
TACAATGAACTGCAGAAAGATAAGATGGCGGAGGCCTACAGTGAGATTGGGAT
GAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCC
TCGCTAA
Amino-acid sequence
(SEQ ID NO: 21)
M A L P V T A L L L P L A L L L H A A R P Q V K L D E T G G G L V Q P G R P M K L S C
V A S G F T F S D Y W M N W V R Q S P E K G L E W V A Q I R D K P Y N Y E T F Y S D
S V K G R F T I S R D D S K S S V Y L Q M N N L G A E D M G I Y Y C T G S F A Y W G
Q G T L V T V S A G G G G S G G G G S G G G G S D I Q M T Q S P A S L S A S E G E T V
T I T C R A S G N I H N Y L A W Y Q Q K Q G K S P Q L L V Y N A K T L P D G V P S R
V S G S G S G T Q Y S L K I N N L Q P E D F G N Y Y C Q Q Y W S T P Y T F G G G S R L
E I K R [symbol] [symbol] [symbol] [symbol] [symbol]
D [symbol] I Y I W A P L A G T C G V L L L S L V I T L Y C K R G R K K L L Y I F K Q P F
M R P V Q T T Q E E D G C S C R E P E E E E G G C E L R V K F S R S A D A P A Y K Q G
Q N Q L Y N E L N L G R R E E Y D V L D K R R G R D P E M G G K P R R K N P Q E
G L Y N E L Q K D K M A E A Y S E I G M K G E R R R G K G H D G L Y Q G L S T A
T K D T Y D A L H M Q A L P P R

### Example 6. CAR Lentivirus Production

Lentivirus was produced by the standard procedure using 293T cells as described in [5]. The inventors generated CD37 CAR constructs inside lentiviral vector cloned into Xba I and EcoR I sites of lentiviral vector. pCD510-CD37-CD28 (or 4-1BB)-CD3 zeta lentiviral CAR construct contained CD37 ScFv-CD28 (or 4-1BB)-CD3zeta insert between the Xba I and Eco RI cloning sites.

The lentiviruses were generated in 293T cells and the titers were established by RT-PCR. Then equal dose of lentiviruses was used for transduction of T cells.

### Example 7. Peripheral Blood Mononuclear Cell (PBMC) Isolation from Whole Blood

Whole blood (Stanford Hospital Blood Center, Stanford, Calif.) was collected from individual or mixed donors (depending on the amount of blood required) in 10 mL Heparin vacutainers (Becton Dickinson). Approximately 10 ml of whole anti-coagulated blood was mixed with sterile phosphate buffered saline (PBS) buffer for a total volume of 20 ml in a 50 ml conical centrifuge tube (PBS, pH 7.4, without Ca²⁺ and Mg²⁺). The layer of cells containing peripheral blood mononuclear cells (PBMC), at the diluted plasma/Ficoll interface was removed, washed twice with PBS, and centrifuged at 200×g for 10 min at room temperature. Cells were counted with a hemocytomter. The PBMC were washed once with CAR-T media (AIM V-AlbuMAX(BSA) (Life Technologies), with 5% AB serum and 1.25 μg/mL amphotericin B (Gemini Bioproducts, Woodland, Calif.), 100 U/mL penicillin, and 100 μg/mL streptomycin) and used for experiments or were frozen at −80 C.

### Example 8. T-Cell Activation from PBMC

Freshly isolated PBMC were washed with 1×PBS (pH7.4, no Ca²⁺/Mg²⁺) and washed once in CAR-T media (AIM V-AlbuMAX(BSA), Life Technologies), with 5% AB serum and 1.25 μg/mL amphotericin B (Gemini Bioproducts, Woodland, Calif.), 100 U/mL penicillin, and 100 μg/mL streptomycin), in the absence of human interleukin-2 (hulL-2) (Invitrogen), at a concentration of 5×10⁵ cells/mL, then wash once in CAR-T medium, without huIL-2, before they were finally resuspended to a final concentration of 5×10⁵ cells/mL in CAR-T medium with 300 U/mL huIL2 (from a 1000× stock; Invitrogen). PBMC and beads were then mixed at a 1:1 bead-to-cell ratio, by transferring 25 μL of beads to 1 mL of PBMC. Desired number of aliquots were dispensed to single wells of a culture plate, and then incubated at 37° C. in the presence of CO₂ for 24 hours before viral transduction.

### Example 9. T-Cell Transduction and Expansion

Following activation of PBMC, cells were incubated for 24 hours at 37° C., 5% CO₂. To each well of 1×10⁶ cells, 5×10⁶ lentivirus and 2 μL/mL of media of Transplus (Alstem, Richmond, Calif.) (a final dilution of 1:500) were added. Cells were incubated for an additional 24 hours before repeating the addition of virus. Cells were then grown in the continued presence of 300 U/ML of IL-2 fresh medium with IL-2 for a period of 12-14 days (total incubation time was dependent on the final umber of CAR-T cells required). Cells concentrations were analyzed every 2-3 days, with media being added at that time to dilute the cell suspension to lx10⁶ cells/mL.

### Example 10. Transduction Verification by FACS

Cells were washed and suspended in FACS buffer (phosphate-buffered saline PBS) plus 0.1% sodium azide and 0.4% BSA). Cells were then divided to 1×10⁶ aliquots.

Fc receptors were blocked with normal goat IgG (Life Technologies). 100 μl of 1:1000 diluted normal goat lgG was added to each tube and incubated on ice for 10 min.

1.0 ml FACS buffer was added to each tube, mixed well and spun down at 300 g for 5 min. Biotin-labeled polyclonal goat anti-mouse-F(ab)₂ antibodies (Life Technologies) were added to detect CD37 ScFv; biotin-labeled normal polyclonal goat IgG antibodies (Life Technologies) was added to serve as an isotype control. (1:200 dilution, reaction volume of 100 μL). Cells were incubated at 4° C. for 25 minutes and washed once with FACS buffer. Cells were suspended in FACS buffer and blocked with normal mouse IgG (Invitrogen) by adding 100 μl 1 1:1000 diluted normal mouse lgG to each tube and incubated on ice for 10 min. Cells were washed with FACS buffer and re-suspended in 100 μl FACs buffer.

The cells were then stained with phycoerythrin (PE)-labeled streptavidin (BD Pharmingen, San Diego, Calif.) and allophycocyanin (APC)-labeled CD3 antibody (eBiocience, San Diego, Calif.).

### Example 11. Real-Time Cytotoxicity Assay

The cytotoxicity was performed using ACEA machine according to manufacturer's protocol as described [5]. The real-time cytotoxicity assay (RTCA) was performed with XCELLigence system.

### Example 12. CD37-CAR-T cells Expressed CD37 scFv

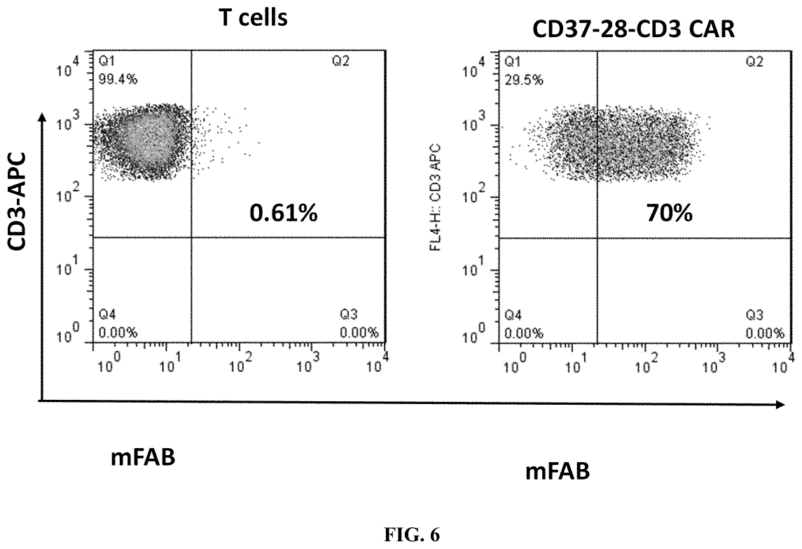

CD37 scFv sequence (Example 1) was inserted with co-stimulating domain CD28 or 41BB and activation domain CD3 zeta inside CAR, and lentiviral CAR were transduced into T cells. The CD37-CAR cells were effectively expanded in vitro (not shown). Mock control with scFv from intracellular protein were generated and used as a negative control in cytotoxicity and cytokine assay. CD37-CAR-positive cells were detected by FACS with mouse anti-FAB antibody that binds to extracellular scFv CAR domain (FIG. 6, right panel). Control non-transduced T cells (FIG. 6, left panel) were not detected by mouse anti-FAB antibody.

### Example 13. CD37-CAR-T Cells Expressed High Cytotoxic Activity Against CD37-Positive Cells Compared to Mock-CAR-T Cells and Non-Transduced T cells and Higher than Against CD37-Negative Cells

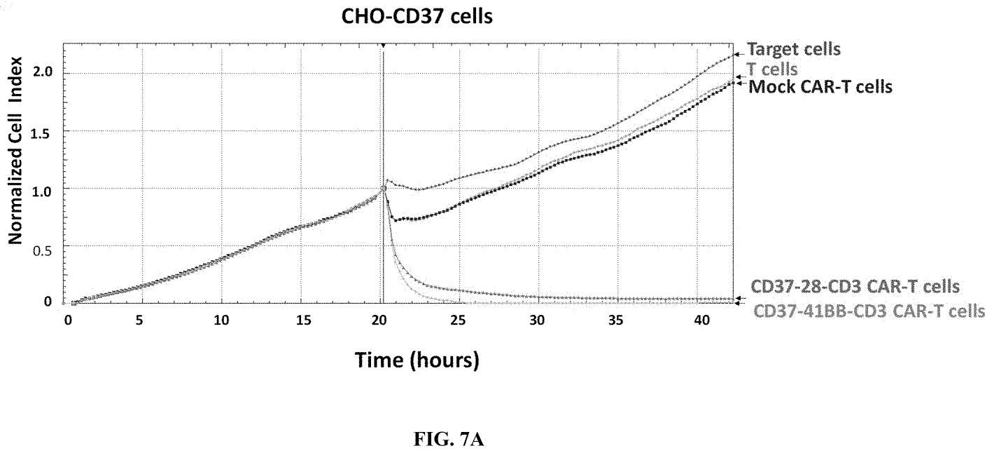

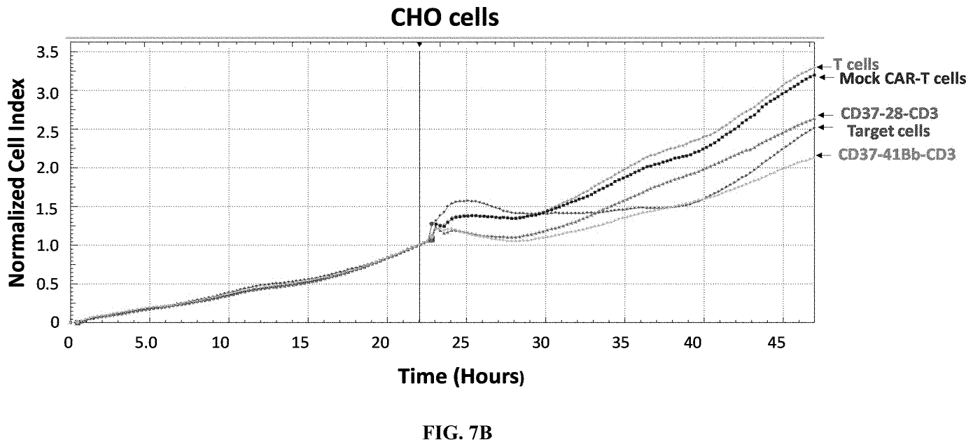

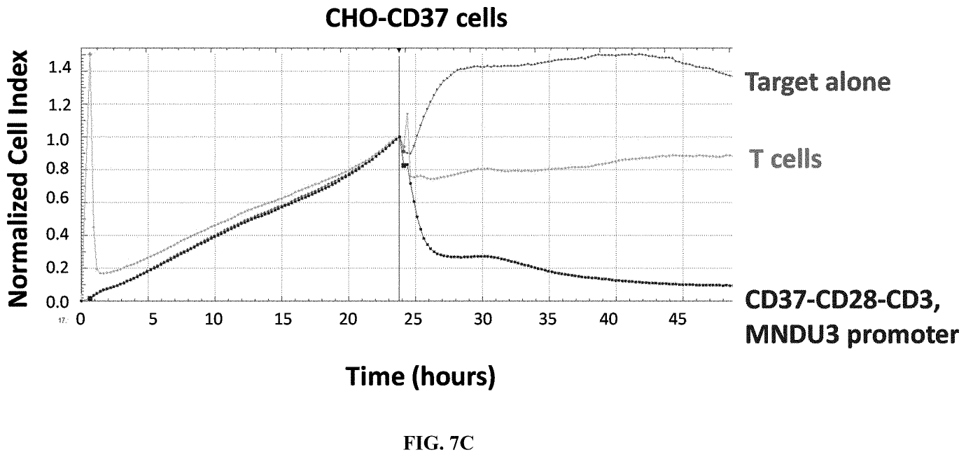

In FIGS. 7A-7B, CD37-CAR were generated using EF-1-lentiviral vector. In FIG. 7C, CD37-CAR was generated using MNDU3 promoter-lentiviral vector.

The RTCA cytotoxicity assay was performed using target CHO-CD37 cells (FIG. 7A) and control CHO cells (FIG. 7B) with T cells, mock CAR-T cells, CD37-CD28-CD3 zeta-CAR-T cells, and CD37-41BB-CD3 zeta-CAR-T cells generated using EF-1 promoter lentivirus. CD37-CAR-T cells specifically killed CHO-CD37 cells (FIG. 7A) but not CHO cells (FIG. 7B).

Similar RTCA activities were observed with CD37-CD28-CD3 zeta-CAR T cells using MNDU3 promoter lentivirus (FIG. 7C).

### Example 14. CD37-CAR-T Cells Secreted High Level of IFN-Gamma Against CD37-Positive Cancer Cells

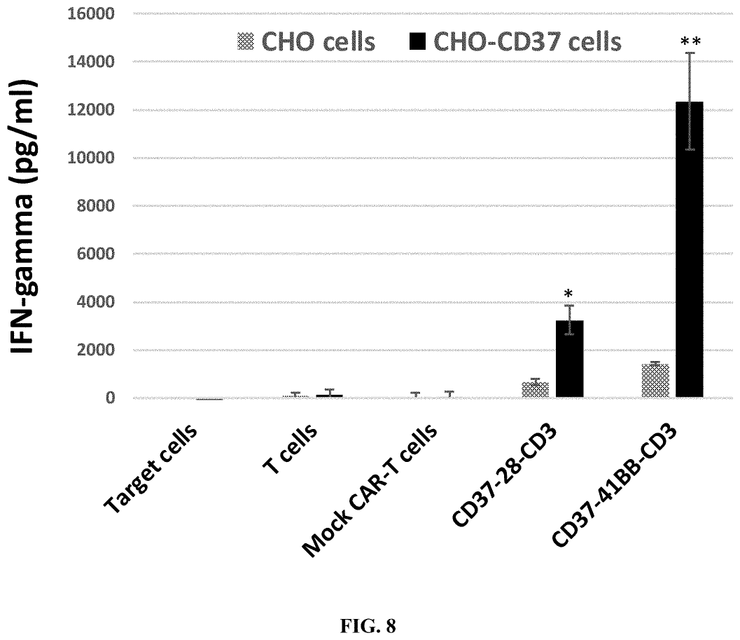

We collected supernatant after CHO-CD37 cancer cells incubated with CD37-CAR-T cells and performed ELISA with a kit from Fisher according to manufacturer's protocol. CD37-CAR-T cells secreted significantly higher level of IFN-gamma against CD37-positive cells than against CD37-negative cells and higher than control T and Mock-CAR-T cells (FIG. 8). FIG. 8. shows high IFN-gamma secretion by CD37-CAR-T cells against CHO-CD37 cells but not against CHO cells.

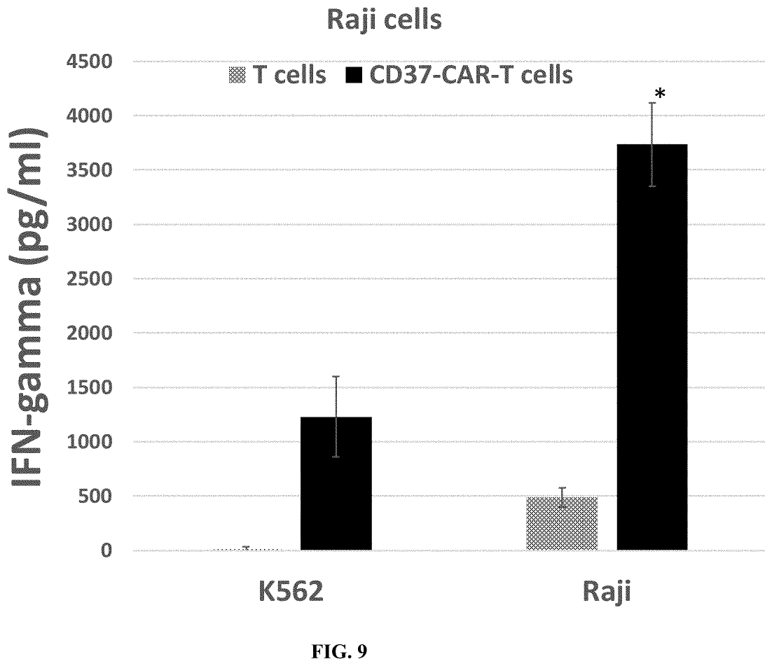

We also incubated CD37-CD28-CAR-T cells with CD37-positive Raji cells and CD37-negative K562 cells and performed ELISA assay with the supernatants to detect secretion of IFN-gamma by CAR-T cells. Significantly higher secretion of IFN-gamma was observed with CD37-CD28-CAR-T cells against Raji target cells than with CD37-CD28-CAR-T cells against K562 cells (FIG. 9). Similar results were observed with CD37-41BB- CAR-T cells (data not shown).

### Example 15. CD37-CAR-T Cells Significantly Decreased Raji Xenograft Tumor Growth In Vivo

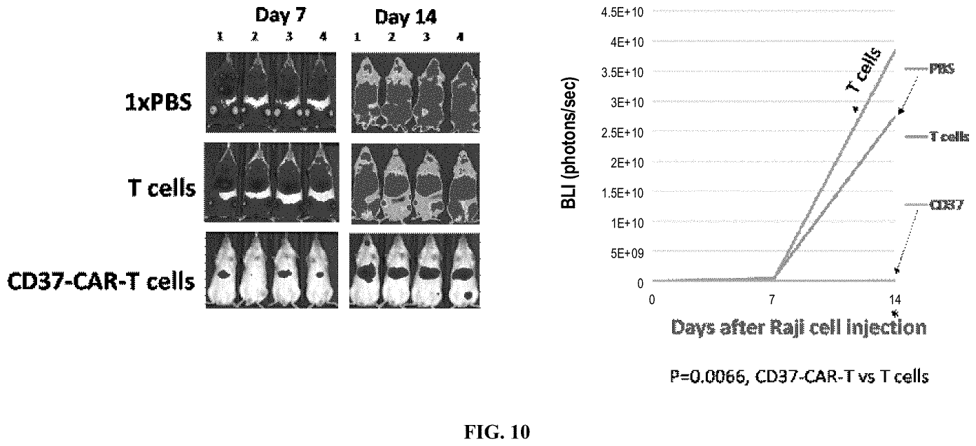

We used NSG mice and injected Raji-luciferase positive cells intravenously into NSG mice and then performed one injection of CD37-CD28-CAR-T cells (Example 5) by i.v in NSG mice at day 1. The bioluminescence signal was significantly decreased in mice treated with CD37-CD28-CAR-T cells than in mice treated with PBS and T cells (FIG. 10), which demonstrated that CD37-CAR-T cells significantly decreased Raji xenograft tumor growth. The mouse body weight was not decreased by CD37-CD28-CAR-T cells suggesting absense of CAR-T cell toxicity (data not shown). CAR-T cells were detected in mice blood showing persistency of CAR-T cells in vivo.

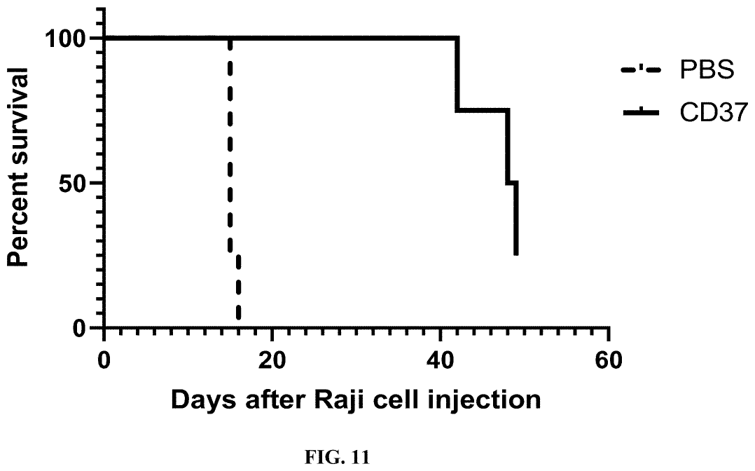

FIG. 11 shows that in Raji xenograft in vivo model, mice treated with CD37-CD28-CAR-T cells prolonged survival of mice compared to mice treated with PBS. Thus, CD37-CAR-T cells suppressed tumor growth and prolonged mice survival in vivo.

### REFERENCES

1. Grupp, S. A., et al. (2013) N Engl J Med 368, 1509-1518.
2. Maus, M. V., et al. (2013) Cancer Immunol Res 1, 26-31.
3. Maus, M. V., et al. (2014) Blood 123, 2625-2635.
4. Boeye, A. (1986) Methods Enzymol 121, 332-340.
5. Berahovich R., et al. (2018) Cancers (Basel), 10 (9).
6. Golubovskaya, V., Wu, L (2016) Cancers, March 15; 8(3).
7. Scarfo, I, et al. (2018) www.bloodjournal.org, DOI 10.1182/blood-2018-04-842708

What is claimed is:

1. A monoclonal anti-human CD37 antibody comprising V_{H} having the amino acid of SEQ ID NO: 3 and V_{L} having the amino acid of SEQ ID NO: 7.

2. A single-chain variable fragment (scFv) comprising V_{H} having the amino acid of SEQ ID NO: 3 and V_{L} having the amino acid of SEQ ID NO: 7.

3. The scFv of claim 2, further comprises a linker in between V_{H} and V_{L}.

4. The scFv of claim 3, which has the amino acid sequence of SEQ ID NO: 8.

5. A chimeric antigen receptor (CAR) comprising from N-terminus to C-terminus:
(i) The scFv of claim 2,
(ii) a transmembrane domain,
(iii) at least one co-stimulatory domains, and
(iv) an activating domain.

6. The CAR of claim 5, wherein the scFv has the amino acid sequence of SEQ ID NO: 8.

7. The CAR according to claim 5, wherein the co-stimulatory domain is CD28 or 4-1BB.

8. The CAR according to claim 5, wherein the activation domain is CD3 zeta.

9. The CAR of claim 5, which has the amino acid sequence of SEQ ID NO: 19.

10. The CAR of claim 5, which has the amino acid sequence of SEQ ID NO: 21.

11. A nucleic acid encoding the CAR of claim 5.

12. T cells modified to express the CAR of claim 5.

13. Natural killer cells modified to express the CAR of claim 5.
